(19) United States
(12) Patent Application Publication
(10) Pub. No.: US 2021/0361859 A1
(43) Pub. Date: Nov. 25, 2021

(54) INTRAVENOUS INFUSION DETECTION DEVICE AND METHOD

(71) Applicant: Quanta Computer Inc., Taoyuan City (TW)

(72) Inventors: Chi-Hsuan LEE, Taoyuan City (TW); Tsung-Ying HSIEH, Taoyuan City (TW)

(21) Appl. No.: 17/017,900

(22) Filed: Sep. 11, 2020

(30) Foreign Application Priority Data
May 21, 2020 (TW) 109116823

(51) Int. Cl. A61M 5/168; G01F 1/66

(52) U.S. Cl. CPC A61M 5/1689; G01F 1/66; A61M 2205/50; A61M 2205/3584; A61M 2205/3317; A61M 2205/3553

(57) ABSTRACT

An intravenous infusion detection device is provided in the invention. The intravenous infusion detection device includes a radar device, a digital signal processing (DSP) device and a controller. The radar device includes a plurality of transmission antennas and a plurality of receiving antennas. The transmission antennas are configured to transmit a plurality of radar signals and the receiving antennas are configured to receive a plurality of reflection signals corresponding to the radar signals. The DSP device transforms the plurality of reflection signals received from the plurality of receiving antennas into a plurality of one-dimensional (1D) waveform diagrams, transforms the plurality of 1D waveform diagrams into a three-dimensional (3D) waveform diagram, and obtains a drip level of a drip bag according to the 3D waveform diagram. The controller obtains the drip level of the drip bag from the DSP device and calculates the flow rate of the intravenous infusion.

### CROSS REFERENCE TO RELATED APPLICATIONS

This Application claims priority of TW Patent Application No. 109116823 filed on May 21, 2020, the entirety of which is incorporated by reference herein.

### BACKGROUND OF THE INVENTION

### Field of the Invention

The invention generally relates to intravenous infusion detection technology, and more particularly, to intravenous infusion detection technology in which the radar device is configured to detect the drip level of intravenous infusion.

### Description of the Related Art

For the intravenous infusion, the blood, medicament, nutrient fluid and other liquid substances can be injected into the vein. The intravenous infusion has more advantages than other medical methods. For example, the route of the intravenous infusion is the fastest way to deliver fluids and medications throughout the patient's body. In addition, the intravenous infusion has batter bioavailability, and therefore, the medication will not be lost in the digestive process and absorption of the digestive system. Furthermore, the intravenous infusion also can be used to provide a route to deliver certain types of medication to the patient's body, when these types of medication cannot be delivered by other methods into patient's body, e.g. the intestinal tract cannot absorb immunoglobulin and propofol.

During the intravenous infusion, the flow rate of the fluids of the intravenous infusion is very important. When fluids are given at a higher rate or a lower rate, some adverse effects might occur in the patient's body. In order to control the flow rate of the fluids, in current intravenous infusion methods, the flow rate of fluids is usually regulated by a manual regulator or by using an electric pump. However, whether the flow rate of the fluids is regulated by a manual regulator or by using an electric pump or not, the regulators, wires and pipes may be usually accidentally touched, as a result, the abnormal flow rate may occur. Therefore, the medical personnel need to check intravenous infusion regularly to ensure both flow rate and delivery of the correct dosage. It causes a waste of human resources, and information about the flow rate is also not reported in real-time.

### BRIEF SUMMARY OF THE INVENTION

An intravenous infusion detection device and method are provided to overcome the problems mentioned above.

An embodiment of the invention provides an intravenous infusion detection device. The intravenous infusion detection device includes a radar device, a digital signal processing (DSP) device and a controller. The radar device includes a plurality of transmission antennas and a plurality of receiving antennas. The transmission antennas are configured to transmit a plurality of radar signals and the receiving antennas are configured to receive a plurality of reflection signals corresponding to the radar signals. The DSP device transforms the reflection signals received from the receiving antennas into a plurality of one-dimensional (1D) waveform diagrams, and it transforms the 1D waveform diagrams into a three-dimensional (3D) waveform diagram. Then, the DSP device obtains a drip level of a drip bag according to the 3D waveform diagram. The controller is coupled to the radar device. The controller obtains the drip level of the drip bag from the DSP device and calculates the flow rate of the intravenous infusion.

In some embodiments, the DSP device is allocated to the radar device or the controller.

In some embodiments, the DSP device adopts an Object Classification algorithm to extract an object which is consistent with the drip bag from the 3D waveform diagram, and obtain the drip level according to the object.

In some embodiments, the Object Classification algorithm is a DSP method or a machine learning (ML) method.

In some embodiments, the controller calculates the flow rate according to a first drip level obtained at a first time point and a second drip level obtained at a second time point.

In some embodiments, the radar signals are step frequency continuous wave (SFCW) signals, the frequency modulated continuous wave (FMCW) signals, pulse-Doppler wave signals or the orthogonal frequency division multiplexing (OFDM) signals.

An embodiment of the invention provides an intravenous infusion detection method. The intravenous infusion detection method is applied to an intravenous infusion detection device. The intravenous infusion detection method includes the steps of transmitting a plurality of radar signals through a radar device of the intravenous infusion detection device; receiving a plurality of reflection signals corresponding to the radar signals through the radar device of the intravenous infusion detection device; transforming the reflection signals received from the receiving antennas into a plurality of one-dimensional (1D) waveform diagrams through a digital signal processing (DSP) device of the intravenous infusion detection device; transforming the 1D waveform diagrams into a three-dimensional (3D) waveform diagram through the DSP device; obtaining a drip level of a drip bag according to the 3D waveform diagram through the DSP device; and obtaining the drip level of the drip bag from the DSP device and calculating a flow rate of the intravenous infusion through a controller of the intravenous infusion detection device.

Other aspects and features of the invention will become apparent to those with ordinary skill in the art upon review of the following descriptions of specific embodiments of intravenous infusion detection device and method.

### BRIEF DESCRIPTION OF THE DRAWINGS

The invention will become more fully understood by referring to the following detailed description with reference to the accompanying drawings, wherein:

FIG. 1 is a block diagram of an intravenous infusion detection device 100 according to an embodiment of the invention;

FIG. 2 is a block diagram of the radar device 110 according to an embodiment of the invention; and

FIG. 3 is a flow chart illustrating an intravenous infusion detection method according to an embodiment of the invention.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

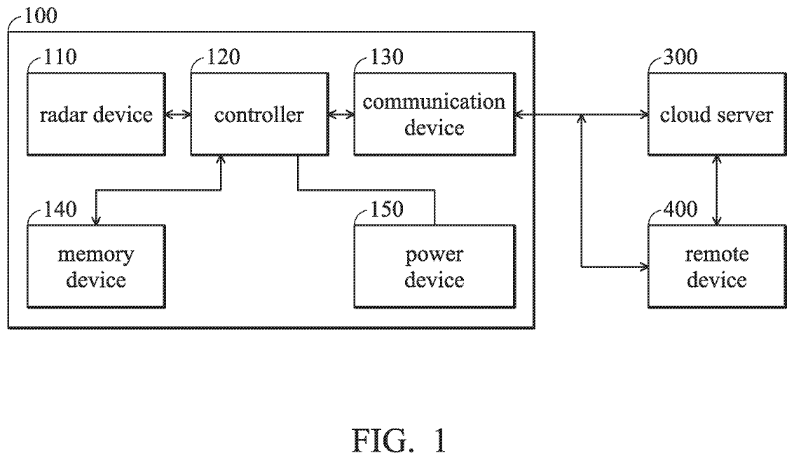

FIG. 1 is a block diagram of an intravenous infusion detection device 100 according to an embodiment of the invention. As shown in FIG. 1, the intravenous infusion detection device 100 comprises a radar device 110, a controller 120, a communication device 130, a memory device 140 and a power device 150. It should be noted that FIG. 1 presents a simplified block diagram in which only the elements relevant to the invention are shown. However, the invention should not be limited to what is shown in FIG. 1. The intravenous infusion detection device 100 may also comprise other elements.

According to the embodiments of the invention, the intravenous infusion detection device 100 can be allocated to the positions where can detect the drip bag.

According to an embodiment of the invention, the controller 120 may be a microcontroller (MCU).

According to embodiments of the invention, the communication device 130 may communicate with a cloud server 300 and a remote device 400 through a wire communication method or a wireless communication method, e.g. Wi-Fi, Bluetooth or Cellular technologies, but the invention should not be limited thereto.

According to embodiments of the invention, the memory device 140 may be a volatile memory (e.g. Random Access Memory (RAM)), or a non-volatile memory (e.g. flash memory, Read Only Memory (ROM)), a hard disk, or a combination of the above memory devices.

According to embodiments of the invention, the power device 150 may provide the power which the intravenous infusion detection device 100 needs to perform the operations of the intravenous infusion detection.

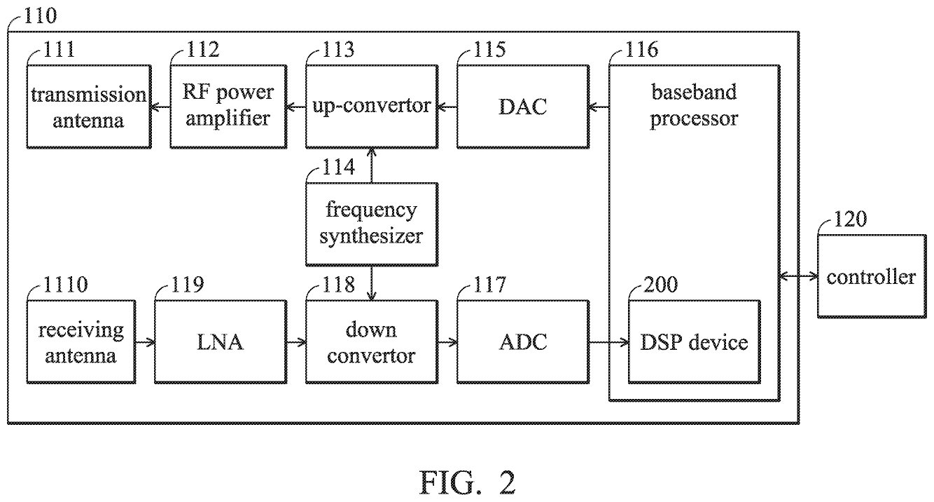

FIG. 2 is a block diagram of the radar device 110 according to an embodiment of the invention. As shown in FIG. 2, the radar device 110 may comprise a plurality of transmission antennas (in order to illustrate the embodiments of the invention simply, FIG. 2 only shows the transmission antenna 111, but the invention should not be limited thereto), radio-frequency (RF) power amplifier 112, an up-convertor 113, a frequency synthesizer 114, a digital-to analog convertor (DAC) 115, a baseband processor 116, an analog-to-digital convertor (ADC) 117, a down convertor 118, a low-noise amplifier (LNA) 119 and a plurality of receiving antennas (in order to illustrate the embodiments of the invention simply, FIG. 2 only shows the receiving antenna 1110, but the invention should not be limited thereto). It should be noted that FIG. 2 presents a simplified block diagram in which only the elements relevant to the invention are shown. However, the invention should not be limited to what is shown in FIG. 2. The radar device 110 may also comprise other elements.

According to the embodiments of the invention, a plurality of transmission antennas (following illustration will use the transmission antenna 111 to illustrate) may be configured to transmit a plurality of radar signals. Each transmission antenna may correspond to different angles and directions. According to an embodiment of the invention, the radar signal may be a step frequency continuous wave (SFCW) signal, the frequency modulated continuous wave (FMCW) signal, a pulse-Doppler wave signal or the orthogonal frequency division multiplexing (OFDM) signal, but the invention should be limited thereto. In addition, in the embodiments of the invention, each transmission antenna may transmit radar signals with different frequencies in different periods. A plurality of receiving antennas (following illustration will use the receiving antenna 1110 to illustrate) may be configured to receive the reflected signals which are the reflected radar signals transmitted by the transmission antennas. In the embodiments of the invention, each receiving antenna may correspond to different angles and directions. In addition, each receiving antenna may receive the reflected signals of the radar signals transmitted by all transmission antennas.

According to the embodiments of the invention, the RF power amplifier 112 may configured to amplify the power of the signals which will be transmitted by the transmission antenna 111. The frequency synthesizer 114 may be configured to provide the oscillation signals with the requirement frequency to the up-convertor 113. The digital-to analog convertor 115 may convert the signals from the baseband processor 116 from the digital signals to the analog signals. The up-convertor 113 may increase the frequency of the output signal of the digital-to analog convertor 115 according to the oscillation signals generated by the frequency synthesizer 114.

According to the embodiments of the invention, the low-noise amplifier 119 may be configured to amplify the signals received by the receiving antenna 1110 for the processions of the next stage of circuits. The frequency synthesizer 114 may be configured to provide the oscillation signals with the requirement frequency to the down convertor 118. The down convertor 118 may reduce the frequency of the output signal of the low-noise amplifier 119 according to the oscillation signals generated by the frequency synthesizer 114. The analog-to-digital convertor 117 may be configured to convert the output signals of the down convertor 118 from the analog signals to the digital signals and transmit the converted signals to the baseband processor 116.

As shown in FIG .2, according to an embodiment of the invention, the baseband processor 116 may comprise a digital signal processing (DSP) device 200. In FIG. 2, the DSP device 200 is allocated to the baseband processor 116, but the invention should not be limited thereto.

The DSP device 200 may transform the signals received from the analog-to-digital convertor 117 into a plurality of one-dimension (1D) waveform diagrams through the inverse fast Fourier transform (IFFT) operation. Then, the DSP device 200 may transform the 1D waveform diagrams into a three-dimension (3D) waveform diagram. Then, the DSP device 200 may adopt an Object Classification algorithm to extract the object which is consistent with the drip bag from the 3D waveform diagram. According to an embodiment of the invention, the Object Classification algorithm may be a digital signal processing (DSP) method. In the DSP method, the DSP device 200 may extract the object which is consistent with the drip bag from the 3D waveform diagram according to the size of the drip bag. According to another embodiment of the invention, the Object Classification algorithm may be a Machine Learning (ML) method. In the ML method, the DSP device 200 may extract the object which is consistent with the drip bag from the 3D waveform diagram according to a machine learning algorithm. After the DSP device 200 obtains the object which is consistent with the drip bag from the 3D waveform diagram, the DSP device 200 may calculate the drip level of the fluids in the object (drip bag), and transmit the drip level of the fluids in the drip bag to the controller 120. According to the embodiments of the invention, the machine learning algorithm may be the Decision Trees algorithm, the Discriminant Analysis algorithm, the Support Vector Machine (SVM) algorithm or the Random Forest algorithm, but the invention should not be limited thereto.

According to the embodiments of the invention, the controller 120 may calculate the flow rate of the intravenous infusion (i.e. the flow rate of the fluids in the drip bag) according to the drip level of the fluids in the drip bag obtained from the DSP device 200. Specifically, the controller 120 may calculate the flow rate according to a first drip level of the fluids in the drip bag obtained at a first time point and a second drip level of the fluids in the drip bag obtained at a second time point. After the controller 120 calculated the flow rate, the related information of the intravenous infusion may be transmitted to the cloud server 300 and/or the remote device 400 (e.g. the monitoring device assigned to medical personnel) through the communication device 130 to perform the intravenous infusion detection. In the embodiments of the invention, the related information of the intravenous infusion may comprise the flow rate of the fluids in the drip bag and the current drip level of the fluids in the drip bag, but the invention should not be limited thereto.

According to an embodiment of the invention, when the controller 120 detect the abnormal flow rate (e.g. the flow rate of the fluids in the drip bag is lower or higher a threshold), the intravenous infusion detection device 100 may transmit an alarm message to the cloud server 300 and/or the remote device 400. When the cloud server 300 and/or the remote device 400 receive the alarm message, the medical personnel can do immediate processes for the abnormal flow rate.

According to another embodiment of the invention, the DSP device 200 may be allocated to the controller 120. In the embodiment, the signal output from the analog-to-digital convertor 117 will be processed by the DSP device of the controller 120. That is to say, in the embodiment, the operations of obtaining the drip level of the fluids in the drip bag and calculating the flow rate of the fluids in the drip bag are performed in the controller 120.

According to another embodiment of the invention, the DSP device 200 may be allocated to the cloud server 300. That is to say, in the embodiment, the intravenous infusion detection device 100 may transmit the signal output from the analog-to-digital convertor 117 to the cloud server 300, and then the operations of obtaining the drip level of the fluids in the drip bag will be performed by the DSP device of the cloud server 300. In addition, in an embodiment of the invention, after the DSP device of cloud server 300 obtains the drip level of the fluids in the drip bag, the processor or controller of the cloud server 300 may directly calculate the flow rate of the fluids in the drip bag according to the drip level of the fluids in the drip bag. Then, the related information (e.g. the flow rate of the fluids in the drip bag and the current drip level of the fluids in the drip bag) of the intravenous infusion may be transmitted to the remote device 400 (e.g. the monitoring device assigned to medical personnel) to perform the intravenous infusion detection. In another embodiment of the invention, after the DSP device of cloud server 300 obtains the drip level of the fluids in the drip bag, the cloud server 300 may transmit the information of the drip level of the fluids in the drip bag to the intravenous infusion detection device 100, and then the controller 120 of the intravenous infusion detection device 100 will calculate the flow rate of the fluids in the drip bag according to the drip level of the fluids in the drip bag. After the controller 120 calculates the flow rate of the fluids in the drip bag, the related information (e.g. the flow rate of the fluids in the drip bag and the current drip level of the fluids in the drip bag) of the intravenous infusion may be transmitted to the cloud server 300 and/or the remote device 400 (e.g. the monitoring device assigned to medical personnel) through the communication device 130 to perform the intravenous infusion detection.

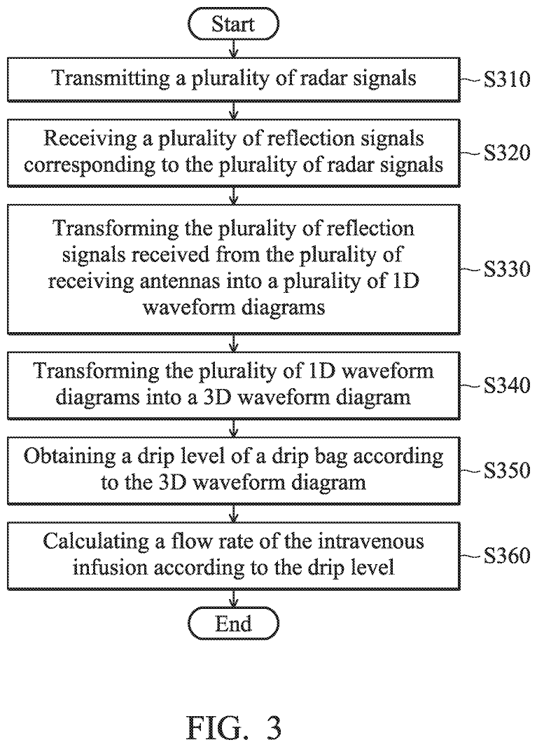

FIG. 3 is a flow chart illustrating an intravenous infusion detection method according to an embodiment of the invention. The intravenous infusion detection method can be applied to the intravenous infusion detection device 100. As shown in FIG. 3, in step S310, the radar device of the intravenous infusion detection device 100 may transmit a plurality of radar signals. In step S320, the radar device of the intravenous infusion detection device 100 may receive a plurality of reflected signals corresponding to the radar signals. According to some embodiments of the invention, the radar signals may be step frequency continuous wave (SFCW) signals.

In step S330, the digital signal processing (DSP) device of the intravenous infusion detection device 100 may transformed the reflected signals to a plurality of one-dimension (1D) waveform diagrams. In step S340, the DSP device of the intravenous infusion detection device 100 may transform the 1D waveform diagrams into a three-dimension (3D) waveform diagram, wherein the reflected signals received by the DSP device have been processed by the radar device.

In step S350, the DSP device of the intravenous infusion detection device 100 may obtain a drip level of the fluids in the drip bag according to the 3D waveform diagram.

In step S360, the controller of the intravenous infusion detection device 100 may calculate the flow rate of the infusion detection according to the drip level of the fluids in the drip bag obtained from the DSP device of the intravenous infusion detection device 100.

According to the embodiments of the invention, step S350 further comprises that the DSP device of the intravenous infusion detection device 100 may adopt an Object Classification algorithm to extract an object which is consistent with the drip bag of the intravenous infusion from the 3D waveform diagram, and obtain the drip level of the fluids in the drip bag according to the object. In some embodiments, the Object Classification algorithm may be a DSP method. In some embodiments, the Object Classification algorithm may be a machine learning (ML) method. The ML algorithm may be the Decision Trees algorithm, the Discriminant Analysis algorithm, the Support Vector Machine (SVM) algorithm or the Random Forest algorithm, but the invention should not be limited thereto.

According to the embodiments of the invention, step S360 further comprises that the controller of the intravenous infusion detection device 100 may calculate the flow rate according to a first drip level of the fluids in the drip bag obtained at a first time point and a second drip level of the fluids in the drip bag obtained at a second time point.

According to the intravenous infusion detection device and method of the invention, the radar device can be used to detect the drip level of the fluids in the drip bag and the flow rate of the intravenous infusion can be calculated according to the drip level of the fluids in the drip bag. According to the intravenous infusion detection device and method of the invention, the flow rate of the fluids in the drip bag and delivery of the correct dosage cam be monitored immediately. When the abnormal flow rate occurs or the fluids in the drip will run out of, the medical personnel can immediately receive the alarm message. Therefore, the medical personnel will not usually go to check the intravenous infusion to ensure the flow rate and delivery of the correct dosage

Use of ordinal terms such as “first”, “second”, “third”, etc., in the disclosure and claims is for description. It does not by itself connote any order or relationship.

The steps of the method described in connection with the aspects disclosed herein may be embodied directly in hardware, in a software module executed by a processor, or in a combination of the two. A software module (e.g., including executable instructions and related data) and other data may reside in a data memory such as RAM memory, flash memory, ROM memory, EPROM memory, EEPROM memory, registers, a hard disk, a removable disk, a CD-ROM, or any other form of computer-readable storage medium known in the art. A sample storage medium may be coupled to a machine such as, for example, a computer/processor (which may be referred to herein, for convenience, as a “processor”) such that the processor can read information (e.g., code) from and write information to the storage medium. A sample storage medium may be integral to the processor. The processor and the storage medium may reside in an ASIC. The ASIC may reside in user equipment. Alternatively, the processor and the storage medium may reside as discrete components in user equipment. Moreover, in some aspects any suitable computer-program product may comprise a computer-readable medium comprising codes relating to one or more of the aspects of the disclosure. In some aspects a computer program product may comprise packaging materials.

The above paragraphs describe many aspects. Obviously, the teaching of the invention can be accomplished by many methods, and any specific configurations or functions in the disclosed embodiments only present a representative condition. Those who are skilled in this technology will understand that all of the disclosed aspects in the invention can be applied independently or be incorporated.

While the invention has been described by way of example and in terms of preferred embodiment, it should be understood that the invention is not limited thereto. Those who are skilled in this technology can still make various alterations and modifications without departing from the scope and spirit of this invention. Therefore, the scope of the present invention shall be defined and protected by the following claims and their equivalents.

What is claimed is:

1. A intravenous infusion detection device, comprising:
a radar device, comprising a plurality of transmission antennas and a plurality of receiving antennas, wherein the plurality of transmission antennas are configured to transmit a plurality of radar signals and the plurality of receiving antennas are configured to receive a plurality of reflection signals corresponding to the plurality of radar signals;
a digital signal processing (DSP) device, transforming the plurality of reflection signals received from the plurality of receiving antennas into a plurality of one-dimensional (1D) waveform diagrams and transforming the plurality of 1D waveform diagrams into a three-dimensional (3D) waveform diagram, and obtaining a drip level of a drip bag according to the 3D waveform diagram; and
a controller, coupled to the radar device and obtaining the drip level of the drip bag from the DSP device and calculating a flow rate of the intravenous infusion.

2. The intravenous infusion detection device of claim 1, wherein the DSP device is allocated to the radar device or the controller.

3. The intravenous infusion detection device of claim 1, wherein the DSP device adopts an Object Classification algorithm to extract an object which is consistent with the drip bag from the 3D waveform diagram, and obtain the drip level according to the object.

4. The intravenous infusion detection device of claim 3, wherein the Object Classification algorithm is a DSP method or a machine learning (ML) method.

5. The intravenous infusion detection device of claim 1, wherein the controller calculates the flow rate according to a first drip level obtained at a first time point and a second drip level obtained at a second time point.

6. The intravenous infusion detection device of claim 1, wherein the plurality of radar signals are step frequency continuous wave (SFCW) signals.

7. A intravenous infusion detection method, applied to an intravenous infusion detection device, comprising:
transmitting, by a radar device of the intravenous infusion detection device, a plurality of radar signals;
receiving, by the radar device of the intravenous infusion detection device, a plurality of reflection signals corresponding to the plurality of radar signals;
transforming, by a digital signal processing (DSP) device of the intravenous infusion detection device, the plurality of reflection signals received from the plurality of receiving antennas into a plurality of one-dimensional (1D) waveform diagrams;
transforming, by the DSP device, the plurality of 1D waveform diagrams into a three-dimensional (3D) waveform diagram;
obtaining, by the DSP device, a drip level of a drip bag according to the 3D waveform diagram; and
obtaining, by a controller of the intravenous infusion detection device, the drip level of the drip bag from the DSP device and calculating a flow rate of the intravenous infusion.

8. The intravenous infusion detection method of claim 7, wherein the DSP device is allocated to the radar device or the controller.

9. The intravenous infusion detection method of claim 7, wherein the DSP device adopts an Object Classification algorithm to extract an object which is consistent with the drip bag from the 3D waveform diagram, and obtain the drip level according to the object.

10. The intravenous infusion detection method of claim 9, wherein the Object Classification algorithm is a DSP method or a machine learning (ML) method.
